(19) United States
(12) Patent Application Publication
(10) Pub. No.: US 2008/0221084 A1
(43) Pub. Date: Sep. 11, 2008

(54) METHOD FOR REDUCING INFARCTION USING VASOPRESSIN ANTAGONIST COMPOUNDS, AND COMPOSITIONS AND COMBINATIONS THEREFOR

(75) Inventors: Yongge LIU, Rockville, MD (US); Junichi KAMBAYASHI, Rockville, MD (US); Hiroyuki FUJIKI, Tokushima-shi (JP); Toyoki MORI, Tokushima-shi (JP)

Correspondence Address: SUGHRUE MION, PLLC, 2100 PENNSYLVANIA AVENUE, N.W., SUITE 800, WASHINGTON, DC, 20037, US

(73) Assignee: OTSUKA PHARMACEUTICAL CO., LTD., Osaka (JP)

(21) Appl. No.: 11/927,153

(22) Filed: Oct. 29, 2007

Related U.S. Application Data
(60) Provisional application No. 60/863,530, filed on Oct. 30, 2006.

(51) Int. Cl. A61K 31/55; A61K 31/551; A61P 9/04

(52) U.S. Cl. 514/215; 514/213.01; 514/220

(57) ABSTRACT

The present invention relates to a method for reducing infarction comprising administering to a patient ion need thereof a therapeutically effective amount of a composition comprising as an active ingredient a vasopressin antagonist compound and to a composition useful therefor. The present invention also relates to a method for reducing infarction comprising administering to a patient in need thereof a therapeutically effective amount of a combination of a vasopressin antagonist compound and a beta-blocker and to combinations useful therefor. The methods, compositions and combinations of the present invention can be used for reducing infarction in the heart (myocardial infarction) and the brain (stroke). The methods, compositions and combinations of the present invention can also be used for the treatment and/or prevention of hypertension, edema, ascites, heart failure, renal function disorder, vasopressin inappropriate secretion syndrome (SIADH), hepatocirrhosis, hyponatremia, hypokalemia, polycystic kidney disease, diabetes, or circulation disorder.

### CROSS-REFERENCE TO RELATED APPLICATION

This application is based on and claims priority from U.S. Provisional Application No. 60/863,530, filed on Oct. 30, 2006 in the US Patent Trademark Office. The disclosure of that provisional patent application is incorporated by reference herein its entirety.

### TECHNICAL FIELD

The present invention relates to a method for reducing infarction comprising administering to a patient a therapeutically effective amount of a composition comprising as an active ingredient a vasopressin antagonist compound and to compositions useful therefor.

The present invention also relates to a method for reducing infarction comprising administering to a patient a therapeutically effective amount of a combination of a vasopressin antagonist compound and a beta-blocker and to combinations useful therefor. The methods, compositions and combinations of the present invention can be used for reducing infarction, including but not limited to infarction in the heart (myocardial infarction (MI)) and the brain (stroke). The methods, compositions and combinations of the present invention can also be used for the treatment and/or prevention of hypertension, edema, ascites, heart failure, renal function disorders, vasopressin inappropriate secretion syndrome (SIADH), hepatocirrhosis, hyponatremia, hypokalemia, polycystic kidney disease, diabetes, or circulation disorder.

### BACKGROUND OF THE INVENTION

According to the most recent statistics from the American Heart Association, there are 1.2 million heart attacks yearly in the U.S. alone (Heart disease and stroke statistics-2006 update, a report from the American Heart Association statistics committee and Stroke statistics subcommittee, American Heart Association). A heart attack happens when a coronary artery supplying blood to the heart is blocked, usually due to the narrowing and closing of the artery as a consequence of athroscrosis and thrombus formation. Heart muscle can only tolerate a short period of oxygen starvation and will die (infarction) in less than 60-120 min. Because heart muscle cells are largely terminately differentiated, they have very limited ability to regenerate. Thus, even though a blocked coronary artery can be reopened with angioplasty and thrombolytic therapy, patients with a heart attack will carry a heart with infarcted tissue for the rest of their life. Because infarcted heart muscle cannot function to pump blood, the patients will have reduced ability to maintain blood supply to the body. Congestive heart failure usually follows and patients may also experience recurrent heart attacks. Patients with heart failure have a reduced mobility, decreased quality of life and shortened life span.

Myocardial ischemia and thus infarction may also occur during elective heart surgery and during heart transplantation. There are currently 5 million people with heart failure and 550,000 new cases each year in the U.S. (Heart disease and stroke statistics-2006 update, a report from the American Heart Association statistics committee and Stroke statistics subcommittee, American Heart Association). There is an unmet medical need to prevent or minimize myocardial infarction during a heart attack or an elective cardiac surgery requiring a period of stopping the coronary flow (ischemia). Such a treatment will improve the likelihood of recovering from a heart attack/ischemia, and limit the possibility of developing heart failure. For patients who already have heart failure, such treatment will also be useful to limit the myocardial infarction from recurrent heart attacks, thus slowing down the progression of heart failure. Treatments that reduce myocardial infarction are anticipated to be life-saving and can reduce hospitalization, enhance quality of life and reduce overall health care costs of high risk patients.

Congestion is one of the most prominent symptoms of heart failure, and is evidenced by fluid retention and volume overload in the patients. Due to the excessive water in the body, patients will have a decreased plasma sodium level (hyponatremia). This is called hypotonic hyponatremia. Vasopressin level is increased in patients with congestive heart failure and plays an important role in the development of hypotonic hyponatremia. Hypotonic hyponatremia has been identified as a risk factor for increased days of hospitalization, morbidity and mortality in patients with heart failure (W H Lee et al., Prognostic importance of serum sodium concentration and its modification by converting-enzyme inhibition in patients with severe chronic heart failure, Circulation 1986; 73:257-267; DS Lee et al., Predicting mortality among patients hospitalized for heart failure: deviation and validation of a clinical model, JAMA 2003; 290:2581-2587; Klein et al., Lower serum sodium is associated with increased short-term mortality in hospitalized patients with worsening heart failure: results from the outcomes of a prospective trial of intravenous milrinone for exacerbations of chronic heart failure (OPTIME-CHE) study, Circulation 2005; 11:2454-2460). In the present invention, while the invention is not limited thereto, a method is described to reduce myocardial infarction in patients with hypotonic hyponatremia. Hypotonic hyponatremia may also occur in other disorders including hypertension, edema, ascites, renal function disorders, vasopressin inappropriate secretion syndrome (SIADH), hepatocirrhosis, hyponatremia, hypokalemia, polycystic kidney disease, diabetes, or circulation disorder. Some of these diseases may increase the likelihood of experiencing a heart attack, stroke or other infarction. Thus this invention will also be useful in patients with these disorders to limit infarction. For example, because the underlying mechanisms of cell death are quite similar in the heart and brain, this invention will also be useful in reducing brain infarction or stroke.

### SUMMARY OF THE INVENTION

The present inventors have found that vasopressin antagonist compounds are effective for reducing infarction in animals.

Thus, the present invention relates to a method for reducing infarction comprising administering a vasopressin antagonist compound, or administering a combination of a vasopressin antagonist compound and a beta blocker to a patient in need thereof. The present invention also relates to combinations of a vasopressin antagonist compound and a beta-blocker. The methods, compositions and combinations of the present invention can be used to reduce/prevent infarction, hypertension, edema, ascites, heart failure, renal function disorder, vasopressin inappropriate secretion syndrome (SIADH), hepatocirrhosis, hyponatremia, hypokalemia, polycystic kidney disease, diabetes, or circulation disorder. The methods, compositions and combinations of the present invention can also be used to reduce/prevent myocardial infarction in the event of elective cardiac surgery requiring a period of stoppage of coronary flow and also to reduce/prevent brain infarction.

Thus, the present invention includes the following various embodiments.

In a first embodiment, the present invention provides a method for reducing infarction comprising administering to a patient in need thereof a therapeutically effective amount of a composition comprising a vasopressin antagonist compound or a pharmaceutically acceptable salt thereof as the active ingredient.

In a second embodiment, the present invention provides a method for reducing infarction according to the first embodiment wherein the infarction is in the heart and/or brain.

In a third embodiment, the present invention provides a method according to the first embodiment, wherein the vasopressin antagonist is a compound represented by formula (I) or a pharmaceutically acceptable salt thereof.

In a fourth embodiment, the present invention provides a method for reducing infarction according to the first embodiment, wherein the vasopressin antagonist is selected from the group consisting of tolvaptan, mozavaptan, conivaptan, lixivaptan, satavaptan, RWJ-351647, RWJ-339489, SSR-149415, YM-222546, YM-471, YM-35471, YM-218, FR-218944, JNJ-17079166, JNJ-17308616, VMAX-367, VMAX-382, VMAX-372, ORG-52186, SRX-251 and pharmaceutically acceptable salts thereof.

In a fifth embodiment, the present invention provides a method for reducing infarction according to the first embodiment, wherein the vasopressin antagonist is a V₂ selective vasopressin antagonist or a V₁/V₂ vasopressin antagonist.

In a sixth embodiment, the present invention provides a method for reducing infarction comprising administering to a patient in need thereof a therapeutically effective amount of a combination of a vasopressin antagonist or a pharmaceutically acceptable salt thereof and a beta-blocker selected from the group consisting of metoprolol, carvediol, propranolol, atenolol, esmolol, sotalol, bisoprolol, labetalol, nadolol and timolol, simultaneously or sequentially.

In a seventh embodiment, the present invention provides a method for reducing infarction according to the sixth embodiment wherein the infarction is in the heart and/or brain.

In an eighth embodiment, the present invention provides a method for reducing infarction according to the sixth embodiment, wherein the vasopressin antagonist is a compound represented by formula (I) or a pharmaceutically acceptable salt thereof.

In a ninth embodiment, the present invention provides a method for reducing infarction according to the sixth embodiment, wherein the vasopressin antagonist is a V₂ selective vasopressin antagonist or a V₁/V₂ vasopressin antagonist.

In a tenth embodiment, the present invention provides a method for reducing infarction according to the sixth embodiment, wherein the vasopressin antagonist is selected from the group consisting of tolvaptan, mozavaptan, conivaptan, lixivaptan, satavaptan, RWJ-351647, RWJ-339489, SSR-149415, YM-222546, YM-471, YM-35471, YM-218, FR-218944, JNJ-17079166, JNJ-17308616, VMAX-367, VMAX-382, VMAX-372, ORG-52186, SRX-251 and a pharmaceutically acceptable salt thereof.

In an eleventh embodiment, the present invention provides a method for reducing infarction according to the tenth embodiment, wherein the vasopressin antagonist is tolvaptan.

In a twelfth embodiment, the present invention provides a method for reducing infarction according to the tenth embodiment, wherein the vasopressin antagonist is mozavaptan hydrochloride.

In a thirteenth embodiment, the present invention provides a method for reducing infarction according to the tenth embodiment, wherein the vasopressin antagonist is conivaptan hydrochloride.

In a fourteenth embodiment, the present invention provides a method for reducing infarction according to the tenth embodiment, wherein the vasopressin antagonist is lixivaptan.

In a fifteenth embodiment, the present invention provides a method for reducing infarction according to the tenth embodiment, wherein the vasopressin antagonist is satavaptan.

In a sixteenth embodiment, the present invention provides a method for reducing infarction according to the sixth embodiment, wherein the vasopressin antagonist is tolvaptan and the beta blocker is metoprolol.

In a seventeenth embodiment, the present invention provides a method for reducing infarction according to the sixth embodiment, wherein the vasopressin antagonist is tolvaptan and the beta blocker is carvediol.

In an eighteenth embodiment, the present invention provides a method for reducing infarction according to the sixth embodiment, wherein the vasopressin antagonist is tolvaptan and the beta blocker is propranolol.

In a nineteenth embodiment, the present invention provides a method for reducing infarction according to the sixth embodiment, wherein the vasopressin antagonist is tolvaptan and the beta blocker is atenolol.

In a twentieth embodiment, the present invention provides a method for reducing infarction according to the sixth embodiment, wherein the vasopressin antagonist is tolvaptan and the beta blocker is esmolol.

In a twenty-first embodiment, the present invention provides a method for reducing infarction according to the sixth embodiment, wherein the vasopressin antagonist is tolvaptan and the beta blocker is sotalol.

In a twenty-second embodiment, the present invention provides a method for reducing infarction according to the sixth embodiment, wherein the vasopressin antagonist is tolvaptan and the beta blocker is labetalol.

In a twenty-third embodiment, the present invention provides a method for reducing infarction according to the sixth embodiment, wherein the vasopressin antagonist is tolvaptan and the beta blocker is nadolol.

In a twenty-fourth embodiment, the present invention provides a method for reducing infarction according to the sixth embodiment, wherein the vasopressin antagonist is tolvaptan and the beta blocker is timolol.

In a twenty-fifth embodiment, the present invention provides a combination of a vasopressin antagonist and a beta blocker selected from the group consisting of metoprolol, carvediol, propranolol, atenolol, esmolol, sotalol, bisoprolol, labetalol, nadolol and timolol.

In a twenty-sixth embodiment, the present invention provides a combination according to the twenty-fifth embodiment wherein the vasopressin antagonist is a compound represented by formula (I) or a pharmaceutically acceptable salt thereof.

In a twenty-seventh embodiment, the present invention provides a combination according to the twenty-fifth embodiment, wherein the vasopressin antagonist is selected from the group consisting of tolvaptan, mozavaptan, conivaptan, lixivaptan, satavaptan, RWJ-351647, RWJ-339489, SSR-149415, YM-222546, YM-471, YM-35471, YM-218, FR-218944, JNJ-17079166, JNJ-17308616, VMAX-367, VMAX-382, VMAX-372, ORG-52186, SRX-251 and pharmaceutically acceptable salts thereof.

In a twenty-eighth embodiment, the present invention provides a combination according to the twenty-seventh embodiment, wherein the vasopressin antagonist is tolvaptan.

In a twenty-ninth embodiment, the present invention provides a combination according to the twenty-seventh embodiment, wherein the vasopressin antagonist is mozavaptan hydrochloride.

In a thirtieth embodiment, the present invention provides a combination according to the twenty-seventh embodiment, wherein the vasopressin antagonist is conivaptan hydrochloride.

In a thirty-first embodiment, the present invention provides a combination according to the twenty-seventh embodiment, wherein the vasopressin antagonist is lixivaptan.

In a thirty-second embodiment, the present invention provides a combination according to the twenty-seventh embodiment, wherein the vasopressin antagonist is satavaptan.

In a thirty-third embodiment, the present invention provides a combination according to the twenty-seventh embodiment, wherein the vasopressin antagonist is tolvaptan and the beta blocker is metoprolol.

In a thirty-fourth embodiment, the present invention provides a combination according to the twenty-seventh embodiment, wherein the vasopressin antagonist is tolvaptan and the beta blocker is carvediol.

In a thirty-fifth embodiment, the present invention provides a combination according to the twenty-seventh embodiment, wherein the vasopressin antagonist is tolvaptan and the beta blocker is propranolol.

In a thirty-sixth embodiment, the present invention provides a combination according to the twenty-seventh embodiment, wherein the vasopressin antagonist is tolvaptan and the beta blocker is atenolol.

In a thirty-seventh embodiment, the present invention provides a combination according to the twenty-seventh embodiment, wherein the vasopressin antagonist is tolvaptan and the beta blocker is esmolol.

In a thirty-eighth embodiment, the present invention provides a combination according to the twenty-seventh embodiment, wherein the vasopressin antagonist is tolvaptan and the beta blocker is sotalol.

In a thirty-ninth embodiment, the present invention provides a combination according to the twenty-seventh embodiment, wherein the vasopressin antagonist is tolvaptan and the beta blocker is labetalol.

In a fortieth embodiment, the present invention provides a combination according to the twenty-seventh embodiment, wherein the vasopressin antagonist is tolvaptan and the beta blocker is nadolol.

In a forty-first embodiment, the present invention provides a combination according to the twenty-seventh embodiment, wherein the vasopressin antagonist is tolvaptan and the beta blocker is timolol.

In a forty-second embodiment, the present invention provides a method for reducing myocardial infarction, comprising administering to a patient in need thereof a therapeutically effective amount of the combination according to the twenty-seventh embodiment.

In a forty-third embodiment, the present invention provides a method for reducing brain infarction, comprising administering to a patient in need thereof a therapeutically effective amount of the combination according to the twenty-seventh embodiment.

In a forty-fourth embodiment, the present invention provides a pharmaceutical composition comprising a vasopressin antagonist and a beta-blocker selected from the group consisting of metoprolol, carvediol, propranolol, atenolol, esmolol, bisoprolol, labetalol, nadolol and timolol.

In a forty-fifth embodiment, the present invention provides a method for treating and/or preventing a condition selected from the group consisting of hypertension, edema, ascites, heart failure, renal function disorder, vasopressin inappropriate secretion syndrome (SIADH), hepatocirrhosis, hyponatremia, hypokalemia, polycystic kidney disease, diabetes, and circulation disorder comprising administering to a patient in need thereof a therapeutically effective amount of a vasopressin antagonist compound.

In a forty-sixth embodiment, the present invention provides a method according to the forty-fifth embodiment, wherein the vasopressin antagonist compound is represented by formula (I).

In a forty-seventh embodiment, the present invention provides a method according to the forty-fifth embodiment wherein the vasopressin antagonist is selected from the group consisting of tolvaptan, mozavaptan, conivaptan, lixivaptan, satavaptan, RWJ-351647, RWJ-339489, SSR-149415, YM-222546, YM-471, YM-35471, YM-218, FR-218944, JNJ-17079166, JNJ-17308616, VMAX-367, VMAX-382, VMAX-372, ORG-52186, SRX-251 and pharmaceutically acceptable salts thereof.

In a forty-eighth embodiment, the present invention provides a method according to the forty-fifth embodiment, wherein the vasopressin antagonist is tolvaptan.

In a forty-ninth embodiment, the present invention provides a method for treating and/or preventing a condition selected from the group consisting of hypertension, edema, ascites, renal function disorders, vasopressin inappropriate secretion syndrome (SIADH), hepatocirrhosis, hyponatremia, hypokalemia, polycystic kidney disease, diabetes, or circulation disorder comprising administering to a patient in need thereof a combination of a vasopressin antagonist compound and a beta-blocker selected from the group consisting of metoprolol, carvediol, propranolol, atenolol, esmolol, sotalol, bisoprolol, labetalol, nadolol and timolol, simultaneously or sequentially.

In a fiftieth embodiment, the present invention provides a method according to the forty-ninth embodiment, wherein the vasopressin compound is represented by formula (I).

In a fifty-first embodiment, the present invention provides a method according to the forty-ninth embodiment wherein the vasopressin compound is tolvaptan.

### BRIEF DESCRIPTION OF THE DRAWINGS

FIG. 1 shows the experimental protocol for Example 1.

FIG. 2 shows risk size data from Example 1.

FIG. 3 shows myocardial infarct size data from Example 1.

FIG. 4 shows correlation between myocardial infarct size and plasma sodium level data from Example 1.

FIG. 5 shows correlation between myocardial infarct size and plasma osmolarity data from Example 1.

### DETAILED DESCRIPTION OF THE INVENTION

An “infarction” generally refers to necrosis of tissue due to upstream obstruction of its arterial blood supply. The lack of oxygenated blood starves the cell to death. An infarction can affect any organ, but occurs more often and faster (<60-120 minutes) in tissue with high energy demand and metabolic activity. These include the heart and the brain.

The term “myocardial infarction” as mentioned herein refers to myocardial necrosis usually resulting from abrupt reduction in coronary blood flow to a segment of myocardium. Myocardium can only sustain a very short period of ischemia (<5 min) without suffering any injury. Reversible injury occurs between 5 to 20 min if blood flow does not resume. A longer period of ischemia will result in permanent injury, i.e., cell death/necrosis/infarction. Because the myocardium (as well as brain) has very limited ability to regenerate, the loss of muscle is therefore permanent. Patients with an infarcted heart will have reduced ability to pump blood, which often results in heart failure and eventual death. About 20% of the patients die within a year of diagnosis of heart failure (Heart disease and stroke statistics-2006 update, a report from the American Heart Association statistics committee and Stroke statistics subcommittee, American Heart Association).

Similarly the term “brain infarction” as mentioned herein refers to brain necrosis usually resulting from abrupt reduction in blood flow to a segment of the brain.

Patients as described herein includes those who have suffered or are at high risk for a heart attack and/or a brain infarction, including, but not limited to those who have been diagnosed with cardiovascular disorders such as coronary artery disease (CAD), systemic hypertension, bicuspid aortic valve, hypertrophic cardiomyopathy, mitral valve prolapse; those who are experiencing or have experienced a heart attack, and/or heart failure (including congestive heart failure (CHF)) or stroke; those who are subject to elective cardiac surgery or brain surgery; and those who have symptoms or conditions related to hypertension, edema, accites, renal function disorders, vasopressin inappropriate secretion syndrome (SIADH), hepatocirrhosis, hyponatremia, hypokalemia, polycystic kidney disease, diabetes or circulation disorder.

In a first embodiment of the present invention, the active ingredient in the method for reducing infarction of the present invention is a vasopressm antagonist compound. Vasopressin antagonist compounds of the present invention include, but are not limited to tolvaptan, mozavaptan, conivaptan, lixivaptan, satavaptan, RWJ-351647, RWJ-339489, SSR-149415, YM-222546, YM-471, YM-35471, YM-218, FR-218944, JNJ-17079166, JNJ-17308616, VMAX-367, VMAX-382, VMAX-372, ORG-52186, SRX-251, or pharmaceutically acceptable salts thereof.

Vasopressin antagonist compounds of the present invention also include benzazepine compounds having activity as a vasopressin antagonist. Such benzazepine compounds have activity at arginine vasopressin (AVP) type 1A (V₁A) and type 2 (V₂) receptors (i.e., V₁/V₂) or are selective for the V₂ receptor. Benzazapine compounds of the present invention include but are not limited to compounds represented by the following formula (I):

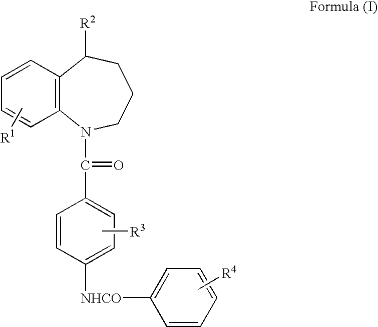

wherein R¹ is a hydrogen atom or a halogen atom, R² is a hydroxy group, or a group of the formula: —NR⁵R⁶ wherein R⁵ and R⁶ are the same or different and are each a hydrogen atom or a lower alkyl group, R³ is a hydrogen atom, a halogen atom, a lower alkyl group, or a lower alkoxy group, R⁴ is a halogen atom, a lower alkyl group or a lower alkoxy group, or a pharmaceutically acceptable salt thereof.

In the description and claims, the groups in the above formula (I) denote the following groups.

The “halogen atom” denotes a fluorine atom, a chlorine atom, a bromine atom, or an iodine atom. The “lower alkyl group” denotes a straight chain or branched chain alkyl group having 1 to 6 carbon atoms, such as methyl, ethyl, propyl, isopropyl, butyl, tert-butyl, pentyl, or hexyl.

The “lower alkoxy group” denotes a straight chain or branched chain alkoxy group having 1 to 6 carbon atoms, such as methoxy, ethoxy, propoxy, isopropoxy, butoxy, tert-butoxy, pentyloxy, or hexyloxy.

The benzazepine compounds of the formula (I) and processes for preparing the same are disclosed in WO 91/05549, U.S. Pat. No. 5,258,510 and U.S. Pat. No. 5,753,677 as well as in the Japanese counterpart JP-A-6-80641, each of which are incorporated by reference in their entirety herein.

The benzazepine compounds of formula (I) of the present invention can readily form a pharmaceutically acceptable acid addition salt with a pharmaceutically acceptable acid. The pharmaceutically acceptable acids include inorganic acids, such as sulfuric acid, hydrochloric acid, phosphoric acid, hydrobromic acid, etc. and organic acids such as acetic acid, p-toluenesulfonic acid, methanesulfonic acid, oxalic acid, maleic acid, fumaric acid, malic acid, tartaric acid, citric acid, succinic acid, benzoic acid, etc.

Among the benzazepine compounds of the formula (I), the compounds having an acidic group can readily form a salt with a pharmaceutically acceptable basic compound. The basic compounds include metal hydroxides such as sodium hydroxide, potassium hydroxide, lithium hydroxide, calcium hydroxide, etc.; alkali metal carbonates or hydrogen carbonates, such as potassium carbonate, sodium carbonate, sodium hydrogen carbonate, potassium hydrogen carbonate, etc.; and alkali metal alcoholates such as sodium methylate, potassium methylate, etc.

The vasopressin antagonist compounds of formula (I) of the present invention are used in the form of a conventional pharmaceutical preparation. The preparation is prepared by using conventional diluents or carriers such as fillers, thickening agents, binders, wetting agents, disintegrators, surfactants, lubricants, and the like. The pharmaceutical preparations can be selected from various forms in accordance with the desired utilities, and the representative forms are tablets, pills, powders, solutions, suspensions, emulsions, granules, capsules, suppositories, injections (solutions, suspensions, etc.), and the like.

In order to form in tablets, there are used well known pharmaceutical carriers such as vehicles (e.g. lactose, white sugar, sodium chloride, glucose, urea, starch, xylitol, mannitol, erythritol, sorbitol, calcium carbonate, kaolin, crystalline cellulose, silicic acid, etc.), binders (e.g. water, ethanol, propanol, simple syrup, glucose solution, starch solution, gelatin solution, carboxylmethyl cellulose, shellac, methyl cellulose, potassium phosphate, polyvinylpyrrolidone, etc.), disintegrators (e.g. dry starch, sodium alginate, agar powder, laminaran powder, sodium hydrogen carbonate, calcium carbonate, polyoxyethylene sorbitan fatty acid esters, sodium laurylsulfate, stearic monoglyceride, starches, lactose, etc.), disintegration inhibitors (e.g. white sugar, stearin, cacao butter, hydrogenated oils, etc.), absorption promoters (e.g. quaternary ammonium base, sodium laurylsulfate, etc.), wetting agents (e.g. glycerin, starches, etc.), adsorbents (e.g. starches, lactose, kaolin, bentonite, colloidal silicates, etc.), lubricants (e.g. purified talc, stearates, boric acid powder, polyethylene glycol, etc.), and the like. Moreover, the tablets may also be in the form of a conventional coated tablet, such as sugar-coated tablets, gelatin-coated tablets, enteric coated tablets, film coating tablets, or double or multiple layer tablets. In the preparation of pills, the conventional carriers can be used and include, for example, vehicles (e.g. glucose, lactose, starches, cacao butter, hydrogenated vegetable oils, kaolin, talc, etc.), binders (e.g. gum arabic powder, tragacanth powder, gelatin, ethanol, etc.), disintegrators (e.g. laminaran, agar, etc.) and the like. In the preparation of suppositories, the conventional carriers can be used and include, for example, polyethylene glycol, cacao butter, higher alcohol, higher alcohol esters, gelatin, semi-synthetic glycerides, and the like. Capsules can be prepared by charging a mixture of the compound of the present invention and the above carriers into hard gelatin capsules, soft capsules or hydroxypropylmethyl cellulose capsules (HPMC capsules) in usual manner. In the preparation of injections, the solutions, emulsions and suspensions are sterilized and are preferably made isotonic with the blood. In the preparation of these solutions, emulsions and suspensions, there are used conventional diluents such as water, ethyl alcohol, macrogol, propylene glycol, ethoxylated isostearyl alcohol, polyoxylated isostearyl alcohol, polyoxyethylene sorbitan fatty acid esters, and the like. In this case, the pharmaceutical preparations may also be incorporated with odium chloride, glucose, or glycerin in an amount sufficient to make them isotonic, and may also be incorporated with conventional solubilizers, buffers, and anesthetizing agents. Moreover, the pharmaceutical preparations may optionally incorporate coloring agents, preservatives, perfumes, flavors, sweetening agents, and other medicaments, if required.

The amount of the vasopressin antagonist compounds of formula (I) to be incorporated into the pharmaceutical composition of the present invention may be selected from a broad range. Usually, the amount preferably in the range of 1 to 70% by weight, more preferably 5 to 50% by weight, based on the weight of the composition.

A suitable method for administration of the compositions of the present invention may be determined in accordance with various forms of preparations, ages, sexes and other conditions of the patients, the degree of severity of diseases, and the like. For example, tablets, pills, solutions, suspensions, emulsions, granules and capsules are administered orally. The injections are intravenously administered alone or together with a conventional auxiliary liquid (e.g. glucose, amino acid solutions), and further are optionally administered alone in intramuscular, intracutaneous, subcutaneous, or intraperitoneal route, if required. Suppositories are administered in intrarectal route.

The dosage of the vasopressin antagonist compounds of the present invention may be selected in accordance with the usage, ages, sexes and other conditions of the patients, the degree of severity of the diseases, and the like. A suitable dose is in the range of 0.1 mg to 1000 mg/body per day, preferably 0.5 mg to 500 mg/body per day, more preferably 1 mg to 100 mg/body per day.

Vasopressin antagonist compounds of the present invention include, but are not limited to tolvaptan, mozavaptan, conivaptan, lixivaptan and satavaptan, or a pharmaceutically acceptable salt thereof. Further, RWJ-351647 and 339489, SSR-149415, YM-222546, YM-471, YM-35471, YM-218, FR-218944, JNJ-17079166 and 17308616, VMAX-367, VMAX-382, VMAX-372, ORG-52186, SRX-251, etc., or pharmaceutically acceptable salts thereof may also be employed as a vasopressin antagonist in the present invention.

In another embodiment, the present invention provides a method for reducing infarction comprising administering to a patient a therapeutically effective amount of a combination of a vasopressin antagonist compound and a beta blocker. The combinations of the invention may include a pharmaceutically acceptable vehicle, carrier or diluent.

Beta blockers are known to reduce myocardial infarction size. Studies have shown that beta blockers reduce epicardial ST-segment elevation and delay cell death in areas of severe ischemia so that smaller areas of necrosis occur subsequent to temporary coronary occlusion. See e.g., Rasmussen, et al., Infarct Size Reduction by Propranolol before and after Coronary Ligation in Dogs, Circulation, Vol. 56, No. 5, p. 794, 1977 and Stephen L. Kopecky, Effect of Beta Blockers, Particularly Carvediol, on Reducing the Risk of Events After Acute Myocardial Infarction, AM J Cardiol 2006: 98:1115-1119. Beta blockers reduce oxygen demand in the heart, and have been shown to slow the progression of heart failure. Beta-blockers are currently standard therapies for heart failure. A combination of the reduction of oxygen demand by beta blockers and the reduction of infarction by vasopressin antagonists will provide additional benefit to prevent and slow down the progression of heart failure. Beta blockers for use in the present invention include but are not limited to metoprolol, carvediol, propranolol, atenolol, esmolol, sotalol, bisoprolol, labetalol, nadolol and timolol.

The selection of the dosage of the vasopressin antagonist compound and the beta blocker is that which can provide relief to the patient as measured by a reduction of infarction and/or amelioration of associated symptoms. As is well known, the dosage of each component depends on several factors such as the potency of the selected specific compound, the mode of administration, the age and weight of the patient, the severity of the condition to be treated, and the like. This is considered to be within the skill of the artisan and one can review the existing literature regarding each component to determine optimal dosing.

In more general terms, one would create a drug combination of the present invention by choosing a dosage of first and second component compounds according to the spirit of the above guidelines.

The presently preferred vasopressin antagonist used according to the invention is Tolvaptan. Tolvaptan, also called, 7-chloro-5-hydroxy-1-[2-methyl-4-(2-methylbenzoyl-amino)benzoyl]-2,3,4,5-tetrahydro-1H-benzazepine, is a selective vasopressin V₂ antagonist. Tolvaptan is represented by the following structure:

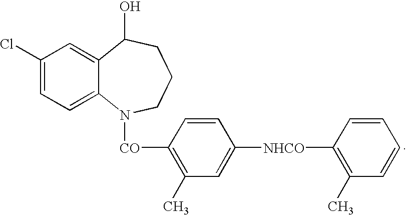

Tolvaptan generates increased, dose-dependent production of diluted urine without altering serum electrolyte balance, and without activation of renin-angiotensin system. Tolvaptan can be used for the treatment and/or prevention of hyponatremia, and hyponatremia and volume overload associated with congestive heart failure (CHF).

Other vasopressin antagonists which can be used include, but are not limited to: mozavaptan (described in U.S. Pat. No. 5,258,510, which is incorporated by reference herein in its entirety), conivaptan (described in U.S. Pat. No. 5,723,606, which is incorporated by reference herein in its entirety), lixivaptan (described in EP 636625 and U.S. Pat. No. 5,516,774, which are each incorporated by reference herein in their entirety) and satavaptan (described in WO971556, which is incorporated by reference herein in its entirety). Further, RWJ-351647 and 339489, SSR-149415, YM-222546, YM-471, YM-35471, YM-218, FR-218944, JNJ-17079166 and 17308616, VMAX-367, VMAX-382, VMAX-372, ORG-52186, SRX-251, etc., may also be employed as vasopressin antagonists in the present invention.

For use in medicine, pharmaceutically acceptable salts may be useful in the preparation of the vasopressin antagonist compounds and the beta-blockers according to the invention. The expression “pharmaceutically acceptable salts” includes both pharmaceutically acceptable acid addition salts and pharmaceutically acceptable cationic salts.

The term “therapeutically effective amount” as used herein refers to a sufficient amount of the compound to reduce infarction, such as for example, myocardial infarction, at a reasonable benefit/risk ratio applicable to any medical treatment.

The specific therapeutically effective dose level for any particular patient will depend upon a variety of factors including the severity of the condition; activity of the specific compound employed; the specific composition employed and the age of the subject. However, some variation in dosage will necessarily occur depending upon the condition of the subject being treated. The person responsible for administration will, in any event, determine the appropriate dose for the individual subject.

The combinations of the present invention can be administered in a standard manner such as orally, parenterally, transmucosally (e.g., sublingually or via buccal administration), topically, transdermally, rectally, via inhalation (e.g., nasal or deep lung inhalation). Parenteral administration includes, but is not limited to intravenous, intraarterial, intraperitoneal, subcutaneous, intramuscular, intrathecal, and intraarticular, or via a high pressure technique, like Powderject™.

For buccal administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate), or welting agents (for example, sodium lauryl sulfate). The tablets can be coated according to methods well known in the art.

Such preparations can also be formulated as suppositories, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions of the present invention can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot preparation. Such long acting formulations can be administered by implantation (for example, subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols.

Alternatively, the compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The combinations of this invention can also be administered in a controlled release formulation such as a slow release or a fast release formulation. Such controlled release formulations of the combinations of this invention may be prepared using methods well known to those skilled in the art. The method of administration will be determined by the attendant physician or other person skilled in the art after an evaluation of the patient's condition and requirements.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates.

The combinations of this invention can also be administered in parenteral form. For parenteral administration, solutions in sesame or peanut oil or in aqueous propylene glycol can be employed, as well as sterile aqueous solutions of the corresponding water-soluble salts. Such aqueous solutions can be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art.

Pharmaceutical compositions according to the invention can contain 0.1%-95% of the therapeutic agents of this invention, preferably 1%-70%. In any event, the composition or formulation to be administered will contain a quantity of therapeutic agent(s) according to the invention in an amount effective to treat the condition or disease of the subject being treated.

The two different compounds of this invention, i.e., the vasopressin antagonist compound and the beta-blocker can be co-administered simultaneously or sequentially in any order, or as a single pharmaceutical composition.

Since the present invention has an aspect that relates to the treatment of the disease/conditions described herein with a combination of active ingredients which can be administered separately, the invention also relates to combining separate pharmaceutical compositions in kit form. An example of such a kit is a so-called blister pack. Blister packs are well known in the packaging industry and are being widely used for the packaging of pharmaceutical unit dosage forms (tablets, capsules, and the like).

The present invention is illustrated in more detail by the following Examples.

### EXAMPLES

### Example 1

The condition of hypotonic hyponatremia was created by infusing a selective vasopressin V₂ agonist, DDAVP, into rats using an osmotic minipump implanted subcutaneously for 14 days in animals with access to only liquid food (a model of SIADH: syndrome of inappropriate antidiuretic hormone secretion). Control rats had the same surgical implantation of an osmotic minipump filled with saline. Rats either received a daily oral administration of tolvaptan or vehicle in an escalating dosing regime. At the end of the 14-day period, rats were subjected to 20 minutes of regional cardiac ischemia and 3 hours of reperfusion, and myocardial infarction was determined.

Tolvaptan was supplied as a spray-dried formulation, which is a mixture of tolvaptan (free form) and hydroxypropylcellulose (HPC-SL) at a ratio of 2:1. Therefore, the amount of tolvaptan (spray-dried form) was 1.5 times that of tolvaptan (free form). Tolvaptan was suspended in 1% hydroxypropylmethylcellulose solution.

The V₂ agonist DDAVP ([deamino-Cys1, D-Arg8]-vasopressin was dissolved in saline (2 μg/mL).

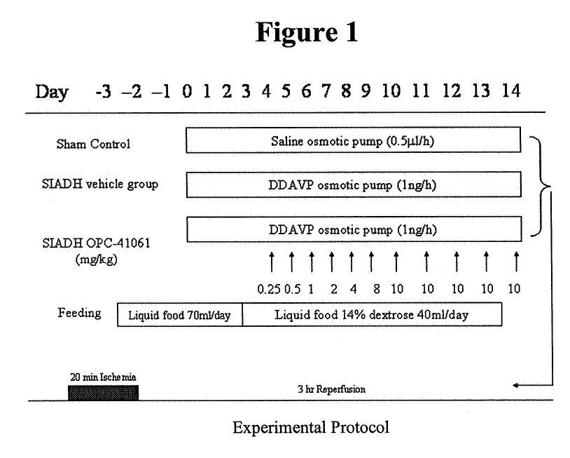

Male Sprague-Dawley rats weighing 150-175 grams were used. Rats had access to feed and tap water. From day −3 (see the timeline in FIG. 1), rats were fed with a customized liquid food (RD-LD89 All-Purpose Liquid Diet Premix, Research Diets, Inc, New Brunswick, N.J.) and no additional food or water was provided. The liquid diet was mixed with water according to manufacturer's instructions. All procedures and husbandry of the animals were in compliance with the Guide for the Care and Use of Laboratory Animals and The Animal Welfare Act, Code of Federal Regulations Title 9, Chapter 1, Subchapter A.

### Groups

| | | | Number of Rats |
|---|---|---|---|
| | | Drug Dose or | Included in Data |
| | Group Name | Concentration | Analysis |
| 1 | Control + Vehicle | Saline infused (0.5 μL/h) & | 8 |
| | | vehicle orally | |
| 2 | DDAVP + Vehicle | DDAVP infused (1 ng/h) & | 10 |
| | | vehicle orally | |
| 3 | DDAVP + | DDAVP infused (1 ng/h) & | 9 |
| | Tolvaptan | Tolvaptan orally (see the | |
| | | figure below) | |

### Experimental Procedures

Rats were acclimated for 3 days to liquid food before the study. Hyponatremia was induced by continuous subcutaneous infusion of 1 ng/h of DDAVP per animal using osmotic minipumps (infusion rate: 0.5 μL/h, ALZET model 2002, DURECT Corporation, Cupertino, Calif.) for 14 days (see FIG. 1). On day 0 when the osmotic minipump was implanted, rats were anesthetized by an intraperitoneal injection of a mixture of ketamine (40 mg/kg) and xylazine (5 mg/kg). The following procedure of implanting minipumps was performed using aseptic techniques. An area 4 cm long and 3 cm wide on the back of the animal (slightly posterior to the scapulae) was shaved and cleaned. A 1 cm cross mid-scapular incision was made. A hemostat was inserted into the incision and a pocket for the pump was created by opening and closing the jaw of the hemostat to spread the subcutaneous tissue. The pump (length 3 cm, diameter 0.7 cm and 0.4 grams) was inserted into the pocket, delivery portal first. The wound was closed with wound clips, and animals were placed on a heating pad for recovery, and eventually returned back to the cage. The sham control rats had the same surgical procedure but with only saline in the minipump.

On day 2 after the surgery, animals were switched to a nutritionally complete liquid food diet containing 14% dextrose (see FIG. 1). No additional water or solid food was provided.

Starting from day 4, an escalating dosing regime of tolvaptan or vehicle was used from 0.25 mg/kg to 10 mg/kg daily. The details of the dosing protocol can be found in FIG. 1. The escalating dosing regimen was used to avoid the potential brain damage such as central pontine myelinolysis associated with a rapid correction of plasma sodium level.

### Dosing Protocol

At the final day of the study (day 14), rats were anesthetized, and the chest was open. Hearts were subjected to 20 minutes of regional ischemia followed by 3 hours of reperfusion.

On day 14, rats were anesthetized with pentobarbital (30 mg/kg) by an intraperitoneal injection. Once anesthesia was induced, a tracheotomy was performed, and the trachea was cannulated and the animal was attached to a respirator. An oxygen supplement was fed into the tracheal line to maintain the pO₂ above 100 mmHg. A cannula was inserted into the right carotid artery to monitor blood pressure and heart rate. The heart was exposed through a left thoracotomy in the fourth intercostal space, and the pericardium opened. A snare was placed around a major branch of the left coronary artery of the animal using a 5-0 suture. The suture ends were passed through a small segment of pliable polyethylene tubing to form a snare. Regional ischemia was induced by pulling the silk tightly through the tubing and clamping the tube with a hemostat. Experiments involved 20 minutes of ischemia and 3 hr reperfusion, which was achieved by releasing the snare. Myocardial ischemia and reperfusion were confirmed by the observation of regional cyanosis and hyperemia, respectively. At the end of the experiment, a Millar miniature pressure catheter was inserted into left carotid artery and advanced into the left ventricle to measure left ventricular pressure. A blood sample was withdrawn to determine blood parameters including plasma sodium and plasma osmolarity at the end of the experiment. The heart was quickly excised for determination of myocardial infarct size.

To determine myocardial infarction, the excised heart was quickly attached to a Langendorff apparatus and flushed with room temperature saline. The coronary snare was pulled again and 1% fluorescent particles (Duke Scientific, Palo Alto, Calif.) were infused into the heart to demarcate the ischemic area. The fluorescent particles lodged in the non-ischemic region of the heart causing the ischemic zone (risk area) to appear as a non-fluorescent perfusion defect. Hearts were then divided into 2 mm slices and stained in 1% triphenyltetrazolium chloride (TTC, Sigma) solution for 15 minutes. The TTC reaction produces a formazan pigment, which caused the tissue to stain as a deep red color. The infarcted area of the heart does not stain, and appears white. Infarct size was determined by planimetry and expressed as a percentage of risk area infarcted.

### Statistical Analysis

Data were expressed as the Mean ±SD (standard deviation). One-way ANOVA with post-hoc Tukey Test (SigmaStat, 3.1) was used and a p<0.05 was considered as significant. To test correlation between infarct size and plasma sodium level or osmolarity, the Pearson Product Moment Correlation test (SigmaStat, 3.1) was used.

### Results

There was no significant difference among the groups on hemodynamics (data not shown).

DDAVP infusion caused plasma sodium to drop to 114±7 mmol/L from 145±2 mmol/L in saline treated rats (p<0.001). Plasma osmolarity was also lower in DDAVP-treated rats (264±6 mOsm/kg vs. 311±5 mOsm/kg in saline infused rats; p<0.001). Thus hypotonic hyponatremia was created in these animals. Tolvaptan effectively corrected this condition as plasma sodium level and osmolarity were returned to 140±3 mmol/L and 304±8 mOsm/kg, respectively, values similar to those in saline infused rats.

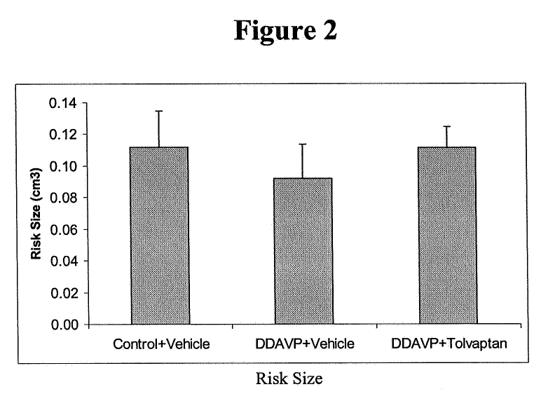

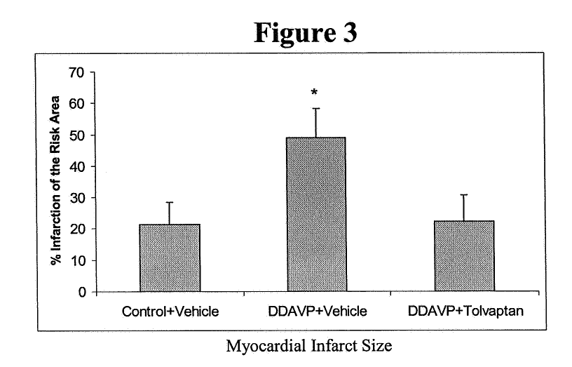

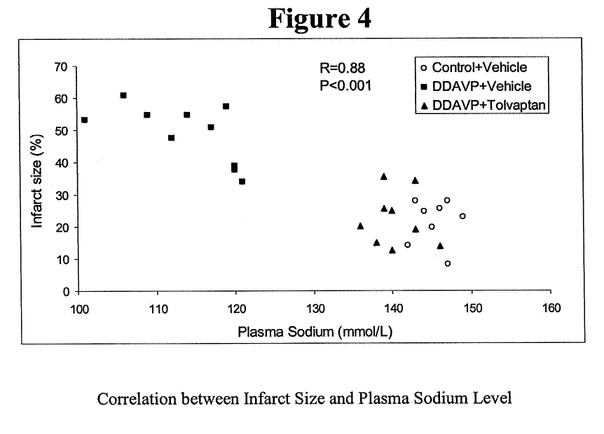

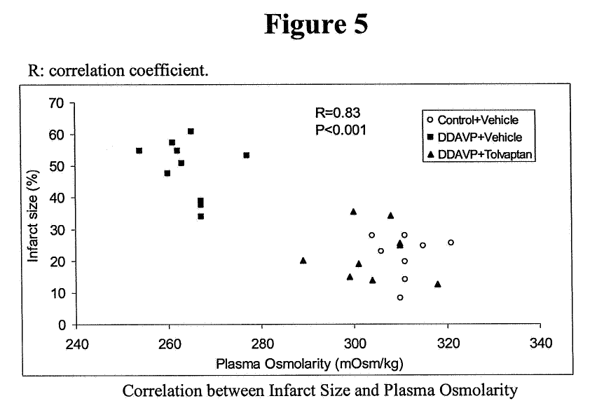

Risk and infarct size data are summarized in FIG. 2 and FIG. 3, respectively. Risk size was similar among the groups. However, infarct size was more than double in DDAVP-infused rats compared with those from saline-infused. Tolvaptan reduced the infarct size in DDAVP-infused rats to a level similar to that in saline-infused rats. To determine whether the size of infarction was correlated with plasma sodium level or osmolarity, the infarct size vs. sodium level or osmolarity was plotted in FIG. 4 and FIG. 5, respectively. A significant negative correlation was detected between infarct size and plasma sodium or osmolarity with a correlation coefficient R value of 0.88 and 0.83, respectively.

### Example 2

While tolvaptan is able to reduce myocardial infarction in rats as shown in Example 1, myocardial infarction nevertheless still occurred in tolvaptan-treated rats, albeit to a much smaller degree as compared with that in vehicle-treated rats. This example describes a method to further reduce the myocardial infarction by a combination of a vasopressin antagonist and a beta blocker. Beta blockers have been shown to reduce myocardial infarction and slow the progression of heart failure (see e.g., Rasmussen, et al., Infarct Size Reduction by Propranolol before and after Coronary Ligation in Dogs, Circulation, Vol. 56, No. 5, p. 794, 1977 and Stephen L. Kopecky, Effect of Beta Blockers, Particularly Carvediol, on Reducing the Risk of Events After Acute Myocardial Infarction, AM J Cardiol 2006: 98:1115-1119) by decreasing the energy demand in the heart. Thus a combination of vasopressin antagonists and beta blockers further reduces the myocardial infarction.

It is shown that tolvaptan can reduce myocardial infarction in hypotonic hyponatremic rats. Hypotonic hyponatremia is prevalent in patients with hypertension, edema, ascites, heart failure, renal function disorder, vasopressin inappropriate secretion syndrome (SIADH), hepatocirrhosis, hyponatremia, hypokalemia, polycystic kidney disease, diabetes, or circulation disorder. Since some of these patients may have underlying coronary artery disease and thus higher risk of heart attacks, tolvaptan and other vasopressin antagonists will be useful to reduce myocardial infarction.

Beta blockers reduce oxygen demand in the myocardium and are standard therapy in heart failure. Decreasing oxygen demand reduces the starvation of ischemic myocardium. Combinational therapies of beta blockers and vasopressin antagonists with the property of reduction myocardial infarction further protects the heart during a heart attack.

Because the underlying mechanisms of cell injury are quite similar between heart and brain, this invention is also useful for the reduction of infarction in stroke.

All patents, patent applications, scientific and medical publications mentioned herein are hereby incorporated by reference in their entirety. It should be understood, of course that the foregoing relates only to preferred embodiments of the present invention and that numerous modifications or alterations may be made by those of ordinary skill in the art without departing from the spirit and scope of the invention as set forth in the appended claims.

What is claimed is:

1. A method for reducing infarction comprising administering to a patient in need thereof a therapeutically effective amount of a composition comprising a vasopressin antagonist compound or a pharmaceutically acceptable salt thereof as the active ingredient.

2. The method of claim 1, wherein the infarction is in the heart and/or brain.

3. The method of claim 1, wherein the vasopressin antagonist is a compound represented by formula (I): 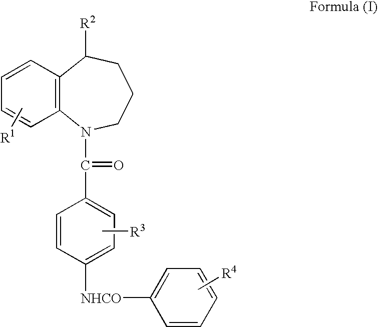
wherein R¹ is a hydrogen atom or a halogen atom, R² is a hydroxy group, or a group of the formula: —NR⁵R⁶ wherein R⁵ and R⁶ are the same or different and are each a hydrogen atom or a lower alkyl group, R³ is a hydrogen atom, a halogen atom, a lower alkyl group, or a lower alkoxy group, R⁴ is a halogen atom, a lower alkyl group or a lower alkoxy group, or a pharmaceutically acceptable salt thereof.

4. The method of claim 1, wherein the vasopressin antagonist is selected from the group consisting of tolvaptan, mozavaptan, conivaptan, lixivaptan, satavaptan, RWJ-351647, RWJ-339489, SSR-149415, YM-222546, YM-471, YM-35471, YM-218, FR-218944, JNJ-17079166, JNJ-17308616, VMAX-367, VMAX-382, VMAX-372, ORG-52186, SRX-251 and a pharmaceutically acceptable salt thereof.

5. The method of claim 1, wherein the vasopressin antagonist is a V₂ selective vasopressin antagonist or a V₁/V₂ vasopressin antagonist.

6. A method for reducing myocardial infarction comprising administering to a patient in need thereof a therapeutically effective amount of a vasopressin antagonist and a beta-blocker selected from the group consisting of metoprolol, carvediol, propranolol, atenolol, esmolol, sotalol, bisoprolol, labetalol, nadolol and timolol, simultaneously or sequentially.

7. The method of claim 6, wherein the infarction is in the heart and/or brain.

8. The method of claim 6, wherein the vasopressin antagonist is a compound represented by formula (I): 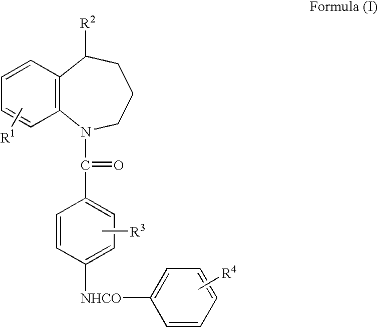
wherein R¹ is a hydrogen atom or a halogen atom, R² is a hydroxy group, or a group of the formula: —NR⁵R⁶ wherein R⁵ and R⁶ are the same or different and are each a hydrogen atom or a lower alkyl group, R³ is a hydrogen atom, a halogen atom, a lower alkyl group, or a lower alkoxy group, R⁴ is a halogen atom, a lower alkyl group or a lower alkoxy group, or a pharmaceutically acceptable salt thereof

9. The method of claim 6, wherein the vasopressin antagonist is a V₂ selective vasopressin antagonist or a V₁/V₂ vasopressin antagonist.

10. The method of claim 6, wherein the vasopressin antagonist is selected from the group consisting of tolvaptan, mozavapatan, conivaptan, lixivaptan, satavaptan, RWJ-351647, RWJ-339489, SSR-149415, YM-222546, YM-471, YM-35471, YM-218, FR-218944, JNJ-17079166, JNJ-17308616, VMAX-367, VMAX-382, VMAX-372, ORG-52186 SRX-251 and a pharmaceutically acceptable salt thereof.

11. The method of claim 5, wherein the vasopressin antagonist is tolvaptan.

12. The method of claim 5, wherein the vasopressin antagonist is mozavaptan hydrochloride.

13. The method of claim 5, wherein the vasopressin antagonist is conivaptan hydrochloride.

14. The method of claim 5, wherein the vasopressin antagonist is lixivaptan.

15. The method of claim 5, wherein the vasopressin antagonist is satavaptan.
